(19) United States
(12) Patent Application Publication
(10) Pub. No.: US 2019/0254828 A1
(43) Pub. Date: Aug. 22, 2019

(54) HIP PROSTHESIS

(71) Applicant: BioMedtrix, LLC, Whippany, NJ (US)

(72) Inventors: Christopher G. Sidebotham, Mendham, NJ (US); Gregory Thomas Van Der Meulen, Ketchum, ID (US)

(73) Assignee: BioMedtrix, LLC, Whippany, NJ (US)

(21) Appl. No.: 16/279,844

(22) Filed: Feb. 19, 2019

Related U.S. Application Data
(60) Provisional application No. 62/632,843, filed on Feb. 20, 2018.

(51) Int. Cl. A61F 2/36; A61F 2/30

(52) U.S. Cl. CPC A61F 2/3662; A61F 2/30749; A61F 2002/3684; A61F 2310/00023; A61F 2002/30322; A61F 2002/30784; A61F 2002/3093; A61F 2002/3625

(57) ABSTRACT

Embodiments of an implant assembly for hip replacement surgery is disclosed. In one embodiment, the implant assembly includes a femoral component comprising a stem member configured to extend into a femur of a patient and a locking member configured to extend through the femoral component to affix the stem member to the femur.

### CROSS-REFERENCE TO RELATED APPLICATION

This application claims the benefit of U.S. Provisional Application No. 62/632,843, filed Feb. 20, 2018, which is incorporated by reference herein.

### FIELD

The present disclosure relates generally to implantable devices and methods for hip replacement surgery.

### BACKGROUND

Joints such as hip joints can become damaged. This damage can be caused by a variety of factors including age, injury, etc. It can also result in pain and limited mobility. Various medical devices and methods have been developed to treat damaged hip joints, including totally or partially replacing the hip joint with prostheses. However, these devices and methods have their shortcomings. As such, there is a need for improved devices and methods for hip replacement procedures.

### SUMMARY

The present disclosure is directed toward methods and apparatuses relating to hip replacement surgery, specifically relating to devices, assemblies, and methods for stabilizing a femoral hip stem.

Certain embodiments of the disclosure concern a hip prosthesis having a femoral component that includes a stem member configured to extend into a femur of a patient. The stem member includes a channel having a first opening and a second opening that is distal to the first opening. The channel has a longitudinal axis that intersects a longitudinal axis of the stem member.

Certain embodiments of the disclosure also concern an assembly including a femoral component and a locking member. The femoral component includes a stem member configured to extend into a femur of a patient. The locking member is configured to extend through the femoral component to affix the stem member to the femur.

Certain embodiments of the disclosure further concern a method for implanting a hip prosthesis. The method includes inserting a stem member of a femoral component into a femur of a patient and inserting a locking member through a channel of the stem member. The locking member is longer than the channel such that a tip portion of the locking member penetrates the femur.

The foregoing and other objects, features, and advantages of the invention will become more apparent from the following detailed description, which proceeds with reference to the accompanying figures.

### BRIEF DESCRIPTION OF THE DRAWINGS

FIG. 1 schematically illustrates the total hip replacement.

FIG. 2A shows different parts of an implant assembly for hip replacement, according to one embodiment.

FIG. 2B shows the assembled implant assembly depicted in FIG. 2A.

FIG. 3 shows a side elevation view of a femoral component of the implant assembly depicted in FIGS. 2A-2B.

FIG. 4A shows the implant assembly depicted in FIG. 2B is fixed within the femoral canal by using bone cement.

FIG. 4B shows the implant assembly depicted in FIG. 2B being press fit into the femur.

FIG. 5A shows a perspective view of a patient's femur and acetabulum together with an implanted hip prosthesis.

FIG. 5B shows a top view of the hip prosthesis and the femur depicted in FIG. 5A.

FIG. 6A shows a side elevation view of a normal hip prosthesis being implanted in a patient.

FIG. 6B shows the head component of the hip prosthesis is dislocated from the acetabular component.

FIG. 7A shows a side elevation view of a femoral component of a hip prosthesis, according to one embodiment.

FIG. 7B shows a top perspective view of the femoral component depicted in FIG. 7A.

FIG. 8 shows a side elevation view of a femoral component of a hip prosthesis, according to another embodiment.

FIG. 9 shows an implant assembly comprising the femoral component depicted in FIG. 7A and a locking member.

FIG. 10A shows one exemplary embodiment of the locking member.

FIG. 10B shows another exemplary embodiment of the locking member.

FIG. 10C shows yet further exemplary embodiment of the locking member.

FIG. 11 shows the locking member being inserted through a channel of the femoral component and penetrating the femur.

FIG. 12A shows a guide member according to one embodiment.

FIG. 12B shows the guide member depicted in FIG. 12A is coupled to the channel of the femoral component to guide the insertion of a drill member.

FIG. 13 is a flow diagram describing a method of implanting a hip prosthesis, according to one embodiment.

### DETAILED DESCRIPTION

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Typical devices and methods for hip replacement procedures have their shortcomings. As such, there is a need for improved devices and methods these procedures. Such devices and methods are disclosed herein.

In total hip replacement, the damaged bone and cartilage are removed and replaced with prosthetic components. Specifically, the articular surfaces of the normal hip joint are replaced with new bearings which are fixed to the bone. These bearings form a ball-and-socket joint and are fixed to the corresponding bones, the hipbone and the femur, using different geometries. For the cup, the implant typically has a spherical type geometry which can mate with a spherically reamed bone. For the femoral component, the implant can have an extended stem for fixation within the canal of the femur following broaching the canal. Fixation of the implants can also be different with some implants being cemented into the bone while others are press fit with porous surfaces for long-term fixation through bone ingrowth.

With press fit applications, the bone must be strong enough to support the implant. Support is primarily afforded by trabecular bone (spongy bone) and secondarily by cortical bone (hard, compact bone). During the first few weeks following the press fit surgery, the bone must keep the implant stable with minimal motion providing the opportunity for the bone to grow into the porous structure of the implants. As the quality of the bone reaches a minimum strength, surgeons make the decision to cement the implant instead of press fitting. However, between the good strength bone and the insufficient strength bone lies a questionable zone of bone quality which may or may not be able to support a press fit application. In those patients, if the preference is to press fit, the stability of the femoral implant may be questionable.

The loading of the acetabular implant is primarily in compression and offers the option for press fitting most of the time. However, the stem of the femoral implant is pressed into a bed of trabecular bone within the femoral canal and in some cases, that bone may not be able to resist torsional, compressive and shear loads. Complications from this inadequacy include subsidence of the stem (i.e., it moves further into the femoral canal) and/or torsional movement of the stem (i.e., it rotates within the canal). In either case, this type of gross motion can lead to poor biomechanics leading to instability and subluxation (the head comes out of the acetabular implant) requiring revision surgery. It can also lead to a fibrous tissue ingrowth, rather than bone ingrowth, into the implant. Thus, it will not provide sufficient long-term stability for the implant. Accordingly, such complications can result in revision surgery, delayed fixation of the implants, delayed return to normal activities, infection and compromised return to normal function.

Thus, it is desirable to improve the design of the femoral implant to allow the surgeon to stabilize the stem of the femoral implant in those cases where there are questions about the strength of the femoral bone.

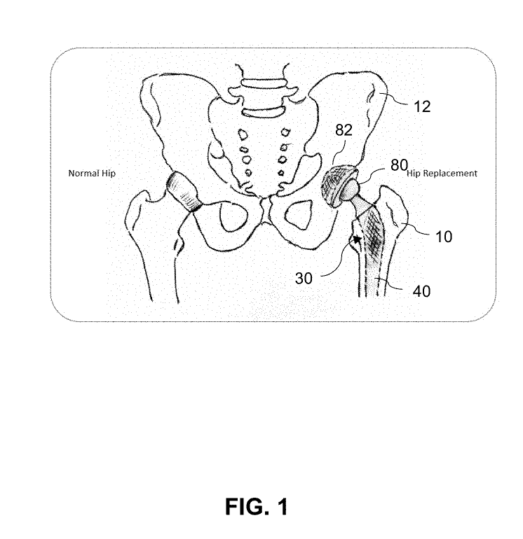

In a hip replacement procedures (also known as hip arthroplasty), the damaged bone and cartilage are removed and replaced with one or more prosthetic components. As illustrated in FIG. 1, the damaged native femoral head can be removed and replaced with a prosthetic femoral component 30 which includes a femoral stem 40 that can be placed into a broached canal in the native femur 10. A prosthetic head component 80, which can, for example, be a metal or ceramic ball, can be placed on the upper part of the femoral component 30. This head component 80 replaces the damaged native femoral head that was removed. The damaged cartilage surface of the native socket (acetabulum) of the pelvis 12 can be removed and replaced with a prosthetic acetabular component 82 such as a metal socket. The acetabular component 82 can be coupled to the pelvis 12 in various ways, including screws, adhesive, and/or other means for coupling.

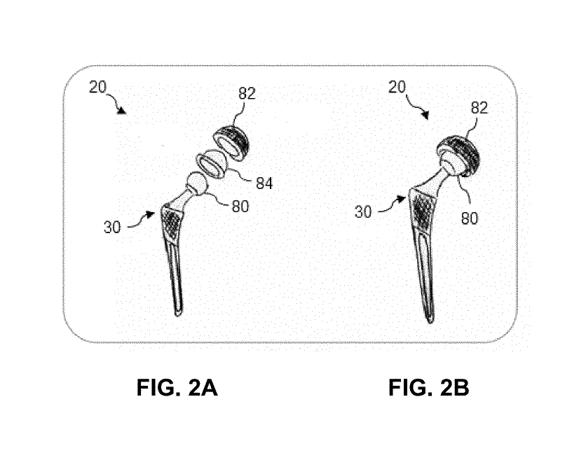

FIGS. 2A-2B shows one exemplary embodiment of a hip prosthesis 20, which includes a femoral component 30, a head component 80, an acetabular component 82 and a liner 84. The liner 84, which can, for example, be made of plastic, ceramic, and/or metal material, can be inserted between the head component 80 and the acetabular component 82 so as to allow for a smooth articulating surface. As shown, the head component 80 is connected to the proximal end portion of the femoral component 30, and the liner 84 is disposed between the head component 80 and the acetabular component 82.

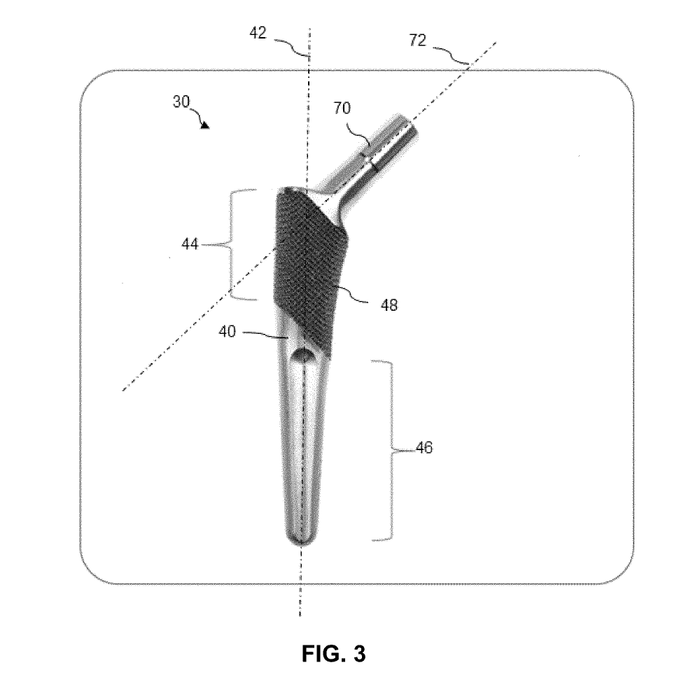

As shown in FIG. 3, the femoral component 30 can include a stem member 40 and a neck member 70 extending from a proximal end portion of the stem member 40. The neck member 70 has a longitudinal axis 72 that intersects a longitudinal axis 42 of the stem member 40. In other words, the neck member 70 is angularly oriented relative to the stem member 40. The proximal end of the neck member 70 is adapted to receive a head component 80.

The femoral component 30 can be formed of various biocompatible materials. In some embodiments, the femoral component 30 can be formed of titanium alloys, such as ASTM F-136 (Ti6Al4V ELI Titanium Alloy). In other embodiments, the femoral component 30 can be formed using other biocompatible materials, such as cobalt chromium, stainless steel, and/or various composite materials or polymers.

The stem member 40 can have a proximal portion 44 and a distal portion 46. The stem member 40 can have a generally tapered shape such that the distal portion 46 has a smaller cross-sectional area than the proximal portion 44.

In some embodiments, the neck member 70 and the stem member 40 can be integrally formed from a single piece of material. In some embodiments, the neck member 70 and the stem member 40 can be made of different materials but are fixedly coupled together to form a unitary piece (e.g., by welding, adhesive, etc.). In other embodiments, the neck member 70 and the stem member 40 can be detachably coupled to each other, such as with fasteners.

The proximal portion 44 can have a porous region 48 configured to promote bone ingrowth, creating mechanical interlocking between the femur 10 and the stem member 40. Such bone ingrowth can improve the long term stability of the femoral component 30 by reducing stress concentrations and bone resorption, as well as improving the torsional strength of the femoral component 30 and reducing the likelihood of the need for revision surgery. Various methods of creating porous surface at the porous region 48 can be employed, as disclosed in U.S. Patent Application Publication No. 2014/0067080, which is incorporated herein by reference.

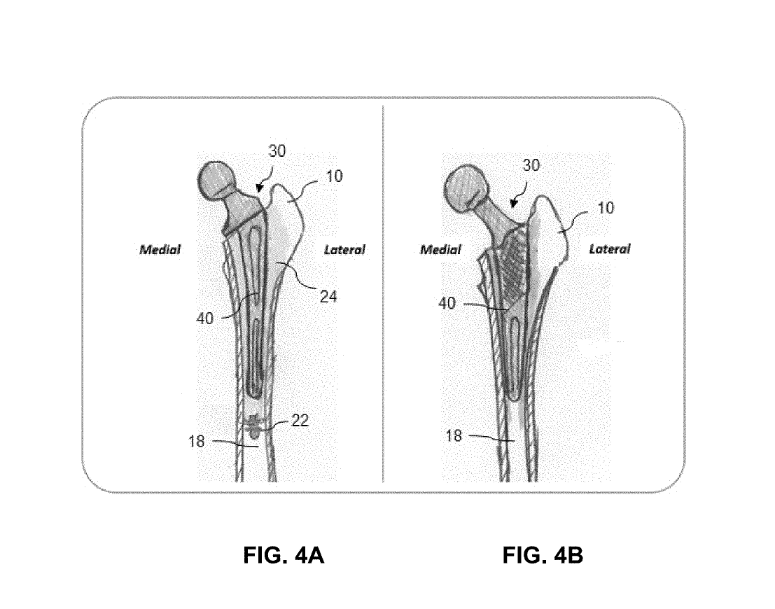

The stem member 40 of the femoral component 30 can be coupled to the femur 10 in various ways. For example, the stem member 40 can be secured in the femoral canal 18 of the femur 10 with an adhesive (e.g., bone cement) and/or a frictional fit (e.g., press fit). FIG. 4A shows, for example, the stem member 40 coupled to the femur 10 with bone cement 24. As shown, an intramedullary plug 22 can be placed distal to the stem member 40, and then the bone cement 24 can be injected into the femoral canal 18 to secure the stem member 40 of the femoral component 30 to the femur 10. FIG. 4B shows the stem member 40 coupled to the femur 10 by press fitting the stem member 40 into the femoral canal 18 of the femur 10. Before implanting the femoral component 30, the femur 10 can be broached or reamed to create the femoral canal 18 that has the same or slightly smaller size than the stem member 40. Thus, the stem member 40 can be wedged into the femoral canal 18 to create an interference or frictional fit for establishing initial stability of the stem member 40.

The stem member 40 needs to resist the loads exerted upon it such that the stem member 40 resists motion between the implant and bone. In some instances, even minimal motion (e.g., greater than about 75-100 microns) between the implant and the bone can cause the implant to be ingrown by fibrous tissue rather than bone tissue. That interface with fibrous tissue can, in some cases, lead to aseptic loosening of the implant, which typically requires revision surgery.

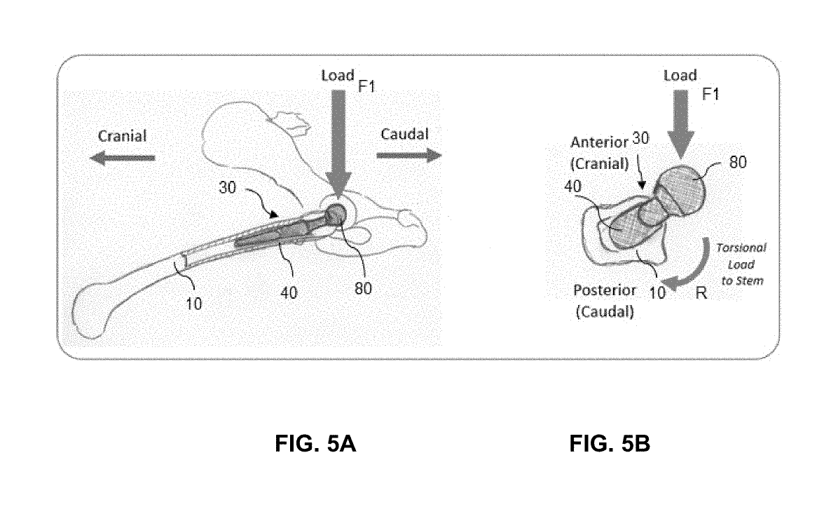

For example, as illustrated in FIGS. 5A-5B, loads (as indicted by arrow F1) to the femoral component 30 caused by rising from a sitting position can create a torsional load to the stem member 40. If the surrounding bone cannot resist such torsion, the stem member 40 may rotate into retroversion (moving posterior or caudally as indicated by arrow R), resulting in possible dislocation of the head component 80 from the acetabular component 82. That type of motion can also lead to aseptic loosening.

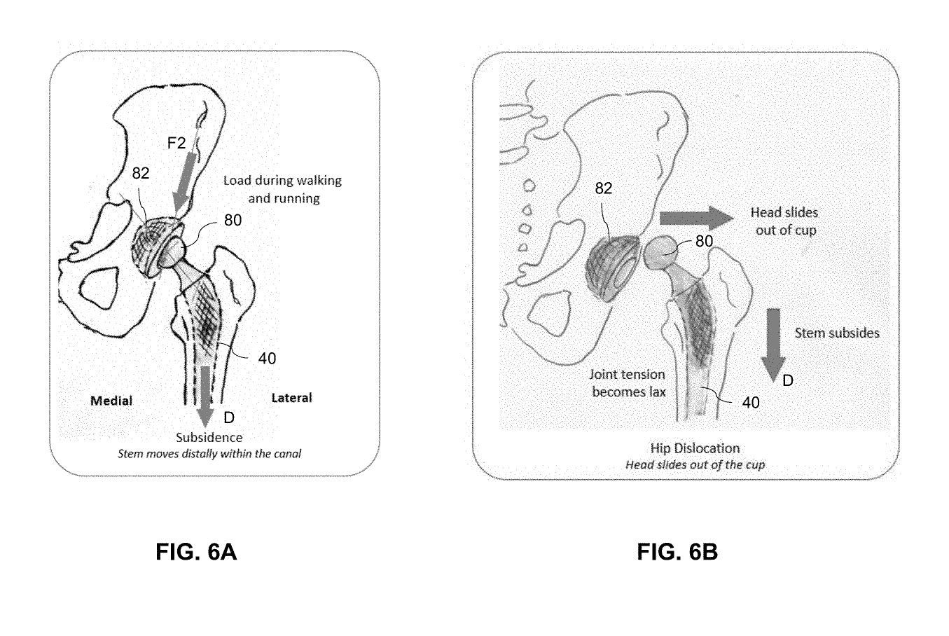

In addition, as shown in FIG. 6A, loads (as indicated by arrow F2) generated during walking and running are more axial directed and can further impact the stem member 40 down (as indicated by arrow D) into the femoral canal 18 unless the bone is capable of resisting such loads. This is termed stem subsidence. If the amount of movement is sufficient, it also can create aseptic loosening of the stem member 40 and dislocation of the head component 80 from the acetabular component 82, as illustrated in FIG. 6B.

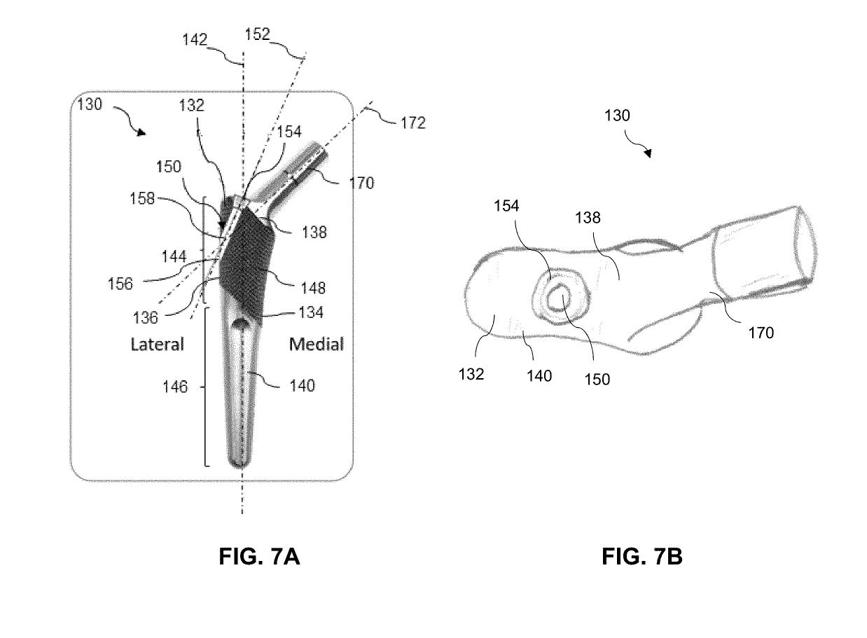

FIGS. 7A-7B show an improved femoral component 130 that can reduce the above-described risk of post-operative complications, according to one embodiment. Specifically, as described more fully below, improved stability of the femoral component 130 can be improved by employing an anchoring mechanism that help to resist torsional and axial loads in the early post-operative period.

Similar to the femoral component 30 described above, the femoral component 130 comprises a stem member 140 and a neck member 170 extending from a proximal end portion 138 of the stem member 140. The neck member 170 has a longitudinal axis 172 that intersects a longitudinal axis 142 of the stem member 140. The stem member 140 can have a generally tapered shape such that a distal portion 146 of the stem member 140 has a smaller cross-sectional area than a proximal portion 144 of the stem member 140. In addition, the proximal portion 144 can have a porous region 148 that is configured to promote bone ingrowth.

Unlike the femoral component 30, the femoral component 130 further comprises a channel 150 extending through the stem member 140. As described more fully below, a locking member can extend through the channel 150 to affix the stem member 140 to the femur 10 in lieu of or in additional to securing the stem member 140 within the femoral canal 18 by frictional engagement and/or adhesive.

The channel 150 has a body portion 158 extending between a first opening 154 (also referred to as a “proximal opening”) and a second opening 156 (also referred to as a “distal opening”), the second opening 156 being distal to the first opening 154. In the embodiment depicted in FIGS. 7A-7B, the channel 150 is oriented in an oblique angle with respect to a longitudinal axis 142 of the stem member 140. That is, a longitudinal axis 152 of the channel 150 extending between the proximal opening 154 and the distal opening 156 intersects the longitudinal axis 142 of the stem member 140.

In the depicted embodiments, the longitudinal axis 152 of the channel 150 is configured to intersect the longitudinal axis 172 of the neck member 170. In other embodiments (not shown), the longitudinal axis 152 of the channel 150 can be generally parallel to the longitudinal axis 172 of the neck member 170.

As depicted in FIGS. 7A-7B, the proximal opening 154 can be located on the proximal end portion 138 of the stem member 140 and is spaced (e.g., laterally) relative to the neck member 170. The distal opening 156 can be located on a lateral side 136 of the stem member 140. As such, the channel 150 generally passes through the lateral portion 132 of the stem member 140.

In some embodiments, the distal opening 156 is generally located at the proximal portion 144 of the stem member 140 such that the distal opening 156 can abut an interior bone tissue adjacent to a lateral cortex of the femur 10 when the stem member 140 extends into the femur 10. As shown in FIG. 7A, the distal opening 156 is located in the porous region 148.

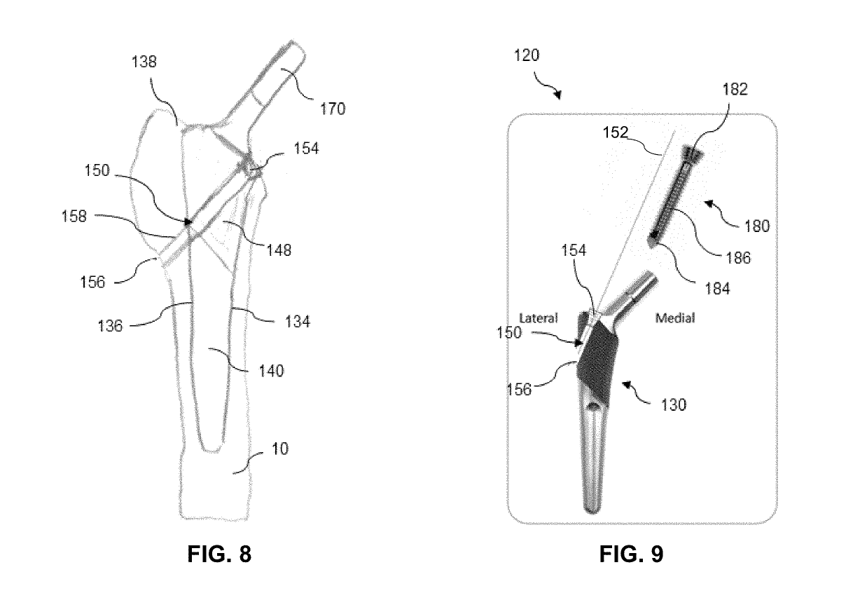

In an alternative embodiment, as shown in FIG. 8, the proximal opening 154 of the channel 150 can be located on the proximal end portion 138 of the stem member 140 and is spaced medially relative to the neck member 170.

Although it is not shown, it should be understood that the channel 150 can be oriented in other different angles with respect to the longitudinal axis 142 so long as it allows a locking member to extend through and affix the stem member 140 to the femur 10. For example, the proximal opening 154 can be located on the lateral side 136 of the stem member 140 whereas the distal opening 156 can be located on a medial side 134 of the stem member 140.

In some embodiments, as illustrated in FIGS. 7A-7B, the proximal opening 154 can be configured as a tapered or conical-shaped countersink recessed from the proximal end portion 138 of the stem member 140. Alternatively, the proximal opening 154 can be configured as a cylindrical flat-bottomed counterbore recessed from the proximal end portion 138 (not shown).

In some embodiments, the proximal opening 154 can be internally threaded. In other embodiments, the proximal opening 154 can have no internal threads. In some embodiments, the body portion 158 of the channel 150 is internally threaded. In some embodiments, the body portion 158 of the channel 150 has no internal threads. Yet in other embodiments, at least one part of the body portion 158 has internal threads and at least another part of the body portion 158 has no internal threads.

FIG. 9 shows an implant assembly 120 that includes the femoral component 130 together with a locking member 180 configured to extend through the channel 150, according to one embodiment.

The locking member 180 includes a head portion 182, a tip portion 184, and a body portion 186 extending between the head portion 182 and the tip portion 184. Generally, the head portion 182 has a larger diameter than the tip portion 184 and the body portion 186.

In some embodiments, the locking member 180 can have a length ranging from about 8 mm to about 80 mm. In some embodiments, the diameter of the body portion 186 of the locking member 180 can range from about 1.5 mm to about 4.5 mm. In some embodiments, the diameter of the channel 150 is about the same or slightly larger than the diameter of the body portion 186 so as to allow insertion of the locking member 180 into the channel 150.

In an exemplary embodiment, the locking member 180 is longer than the channel 150 inside the stem member 140. Thus, when the locking member 180 is disposed in the channel 150 and the head portion 182 is aligned with the proximal opening 154, the tip portion 184 can protrude beyond the distal opening 156.

In some embodiments, the head portion 182 of the locking member 180 is configured to mate with the proximal opening 154 of the channel 150. For example, when the proximal opening 154 is internally threaded, the head portion 182 can be externally threaded such that the head portion 182 can be threadably coupled to the proximal opening 154. Optionally, a washer (not shown) may be inserted into the first opening 154 before inserting the locking member 180 into the channel 150.

Alternatively, the head portion 182 can mate with the proximal opening 154 by other means. For example, a non-threaded head portion 182 can be detachably coupled to a non-threaded proximal opening 154 by means of interference fit, snap fit, or a tongue-and-groove locking mechanism, etc.

The tip portion 184 can be adapted to penetrate bone of different strength. For example, the tip portion 184 can be configured to have various shapes such as oval, cone, half-tipped, plain cup, flat, etc. The head portion 182 can be configured to be compatible with different screw types such as slotted, Phillips, hex, Torx, etc.

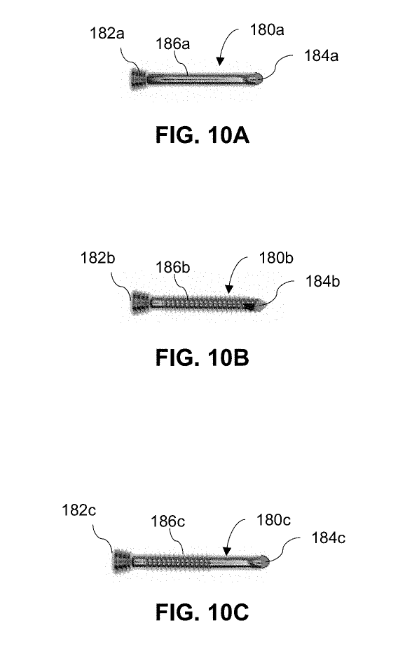

FIG. 10A-10C shows three exemplary embodiments of locking members 180a, 180b, and 180c, respectively. The locking member 180a is configured as a locking bolt, which includes a threaded head portion 182a, an oval-shaped tip portion 184a, and a smooth body portion 186a. The locking member 180b is configured as a locking screw, which includes threaded head portion 182b, a cone-shaped tip portion 184b, and a threaded body portion 186b. The locking member 180c is configured as a locking bolt-screw, which includes a threaded head portion 182c, an oval-shaped tip portion 184c, and a partially threaded body portion 186c.

In the embodiment depicted in FIG. 10C, the proximal part of the body portion 186c adjacent to the head portion 182c is threaded and the distal part of the body portion 186c adjacent to the tip portion 184c is smooth. In other embodiments, the proximal part of the body portion 186c adjacent to the head portion 182c can be smooth and the distal part of the body portion 186c adjacent to the tip portion 184c can be threaded. Alternatively, the body portion 186c can have one or more threaded regions alternate with one or more non-threaded regions.

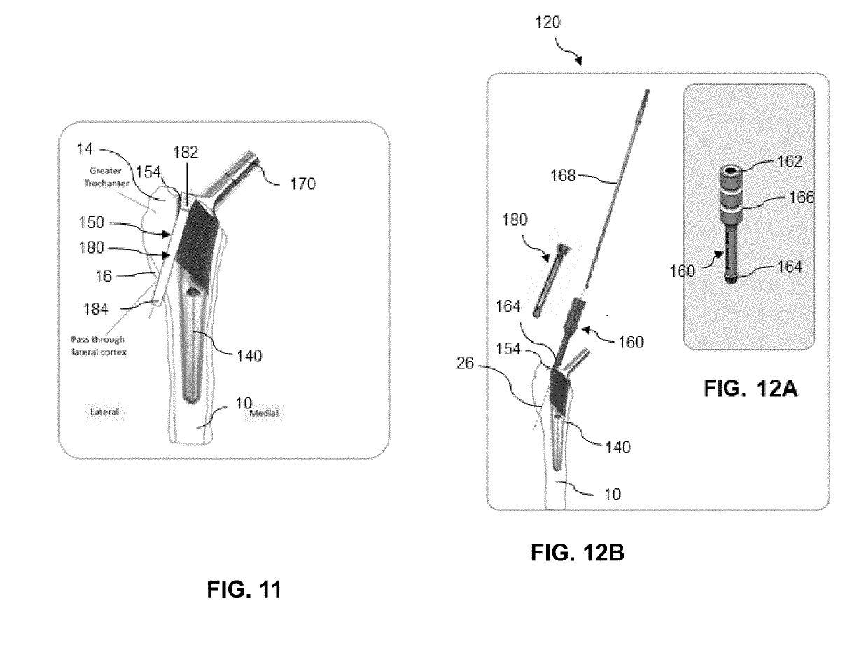

FIG. 11 shows the locking member 180 extends through the channel 150 and affixes the stem member 140 to the femur 10. As shown, after inserting the locking member 180 into the channel 150, the tip portion 184 of the locking member 180 can protrude outside of the distal opening 156 and penetrate the femur 10. The tip portion 184 can advance into and/or through the femur 10 until the head portion 182 of the locking member 180 aligns and/or couples with the proximal opening 154 of the channel 150.

In the depicted embodiment, the locking member 180 passes through the posterior (caudal) portion of the great trochanter 14 and the tip portion 184 protrude out of the lateral cortex 16 of the femur 10.

Optionally, a restraining member (not shown), such as a nut or a washer, can be attached to the tip portion 184 to prevent movement of the locking member 180 relative to the femur 10. Other restraining mechanisms, e.g., attaching hinged flaps to the tip portion 184 to form a toggle bolt, can also be employed to securely hold the locking member 180 to the femur 10.

In other embodiments (not shown), the locking member 180 is configured to penetrate the posterior (caudal) portion of the great trochanter 14, but the tip portion 184 does not protrude out of the lateral cortex 16.

Optionally, the locking member 180 can be configured to be expandable after penetrating the femur 10 so as to further secure the locking member 180 in place. In one exemplary embodiment, the body portion 186 of the locking member 180 can include one or more self-expandable barbs or wings, e.g., being made of shape-memory alloy (e.g. Nitinol), that can change from a collapsed configuration and to an expanded configuration after implantation.

In some embodiments, the locking member 180 can have areas that are coated with a porous material that is configured to facilitate bone ingrowth, which can further secure the locking member 180 within the femur 10.

Optionally, after affixing the stem member 140 to the femur 10 with the locking member 180, the proximal opening 154 of the channel 150 can be covered with a cap (not shown) or sealed with a biocompatible material to cover the head portion 182 of the locking member 140.

As illustrated in FIGS. 12A-12B, the implant assembly 120 can further include a guide member 160 that is configured to matingly engage the channel 150. In some embodiments, the guide member 160 has a central lumen or passage 162 that is configured to pass through a drill member 168. The guide member 160 can be configured to engage with the channel 150 so as to longitudinally align the passage 162 with the channel 150, thereby allowing the drill member 168 to extend through the passage 162 and the channel 150.

In one embodiment, a distal end portion 164 of the guide member 160 can be configured to mate with the proximal opening 154 of the channel 150. For example, the distal end portion 164 of the guide member 160 can be tapered and externally threaded so as to mate with the internal threads of the proximal opening 154. In another example, the distal end portion 164 of the guide member 160 can snap fit into the proximal opening 154.

In other embodiments, the distal end portion 164 of the guide member 160 can be configured to engage with the body portion 158, or even the distal opening 156, of the channel 150. For example, the distal end portion 164 of the guide member 160 can have a smaller diameter than the body portion 158 so that the guide member 160 can be inserted into the body portion 158 channel 150. The proximal end portion 166 of the guide member 160 can have a larger diameter than the body portion 158 so that the proximal end portion 166 can remain outside the channel 150 for receiving the drill member 168.

After inserting the drill member 168 into the channel 150 through the guide member 160, a hand drill can be employed to drive the drill member 168 into the femur 10. Thus, a hole 26 can be created (pre-drilled) in the femur 10 to facilitate insertion of the locking member 180.

In one exemplary embodiment, the hole 26 has a diameter that is smaller than that of the locking member 180. After removing the drill member 168 and the guide member 160, the locking member 180 can extend through the channel 150 and be screwed into the hole 26, thus affixing the stem member 140 to the femur 10.

In alternative embodiments, the hole 26 can have a diameter that is comparable or slightly larger than that of the locking member 180. For example, an expandable anchor plug (not shown) can be inserted into the hole 26, and then the locking member 180 can be inserted through the anchor plug, thereby expanding the anchor plug to securely anchor the locking member 180 within the hole 26. In another example, the locking member 180 can be expandable. For example, the locking member 180 can be configured as a molly fastener that includes one or more sleeves that can be expanded after being inserted into the hole 26.

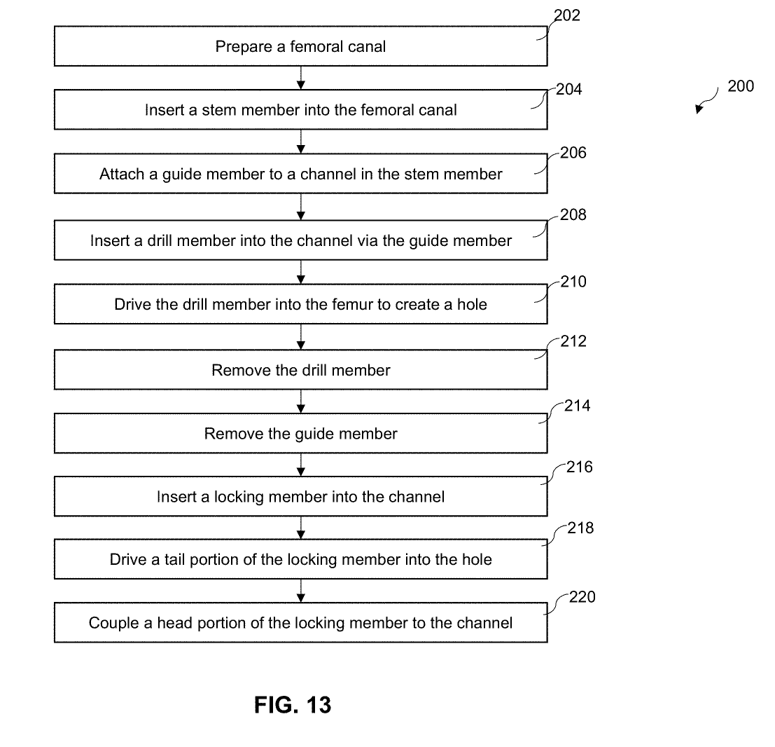

FIG. 13 illustrates an exemplary process 200 of implanting the femoral component 130 of the implant assembly 120. The process 200 can comprise various steps, including preparing a femoral canal 18 in the femur 10 (process block 202), and inserting the stem member 140 of the femoral component 130 into the femoral canal 18 at 204. A guide member 160 can be attached or coupled to the channel 150 located on the stem member 140 (process block 206). A drill member 168 can be inserted into the channel 150 through the guide member 160 (process block 208). The process 200 can also include driving the drill member 168 into the femur to create a hole 26 (process block 210). After removing the drill member 168 and the guide member 160 (process blocks 212 and 214, respectively), a locking member 180 can be inserted into the channel 150 (process block 216). The process 200 can further include driving the tip portion 184 of the locking member 180 into the hole 26 to fasten the stem member 140 with the femur 10 (process block 218). The advancement of the tip portion 184 can be continued until the head portion 182 of the locking member 180 is coupled to the proximal opening 154 of the channel 150 (process block 220).

In certain embodiments, one or more of the steps shown in process 200 may be combined, and/or performed in different sequences. In certain embodiments, one or more of the steps of the process 200 may be optional. In other embodiments, one or more additional steps can be performed before and/or after one or more the steps of the process 200. For example, instead of using a guide member 160 to guide the insertion of the drill member 168, in certain embodiments, the drill member 168 can be inserted directly into the channel 150 for drilling the hole 26. In another example, the step of predrilling the hole 26 may be omitted. Instead, the locking member 180 can be directly drilled into the femur 10 for affixing the stem member 140 to the femur 10.

### GENERAL CONSIDERATIONS

It should be understood that the disclosed embodiments can be adapted to deliver and implant prosthetic devices in orthopedic surgical procedures other than hip arthroplasty, such as shoulder arthroplasty, knee arthroplasty, elbow arthroplasty, ankle arthroplasty, etc.

Although the inventive subject matter disclosed herein is described in the context of implanting a hip prosthesis in a patient, it should be understood that the same general principles and various embodiments disclosed above can be adapted to deliver and implant the hip prosthesis in non-human animals, such as canine, swine, bovine, equine, etc.

As used herein, with reference to the stem member and the neck member, “proximal” refers to a position, direction, or portion of a device that is closer to an acetabulum of a pelvis, while “distal” refers to a position, direction, or portion of a device that is further away from the acetabulum. The terms “longitudinal” and “axial” refer to an axis extending in the proximal and distal directions, unless otherwise expressly defined.

As used herein, the term “porous” means a structure having one or more openings, gaps, or other such surfaces that allow bone to grow into the structure and mechanically interlock with the structure. “Bone ingrowth” refers to the growing of bone tissue into a porous structure in a manner that allows the bone to interlock with the structure.

As used herein, the term “smooth” means a structure lacking in openings, gaps, or other such surfaces that would allow bone to grow into the structure.

As used in this application and in the claims, the singular forms “a,” “an,” and “the” include the plural forms unless the context clearly dictates otherwise. Additionally, the term “includes” means “comprises.” Further, the terms “coupled” and “connected” generally mean physically, mechanically, chemically, magnetically, and/or electrically linked and do not exclude the presence of intermediate elements between the coupled or connected items absent specific contrary language.

As used herein, the term “approximately” and “about” means the listed value and any value that is within 20% of the listed value. For example, “about 8 mm” means any value between about 6.4 mm and about 9.6 mm, inclusive.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” and the like. Such terms are used, where applicable, to provide some clarity of description when dealing with relative relationships, particularly with respect to the illustrated embodiments. Such terms are not, however, intended to imply absolute relationships, positions, and/or orientations. As used herein, “and/or” means “and” or “or”, as well as “and” and “or”.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

In view of the many possible embodiments to which the principles of the disclosure may be applied, it should be recognized that the illustrated embodiments are only examples and should not be taken as limiting the scope of the claimed subject matter. Rather, the scope of the claimed subject matter is defined by the following claims and their equivalents.

1. A hip prosthesis comprising:
a femoral component comprising a stem member configured to extend into a femur of a patient,
wherein the stem member comprises a channel having a first opening and a second opening that is distal to the first opening, and
wherein the channel has a longitudinal axis that intersects a longitudinal axis of the stem member.

2. The hip prosthesis of claim 1, wherein the femoral component further comprises a neck member extending from a proximal end portion of the stem member, and the neck member has a longitudinal axis that intersects the longitudinal axis of the stem member.

3. The hip prosthesis of claim 2, wherein the first opening is located on the proximal end portion of the stem member and is spaced lateral relative to the neck member.

4. The hip prosthesis of claim 1, wherein the second opening is located on a lateral side of the stem member.

5. The hip prosthesis of claim 4, wherein the second opening is located at a proximal portion of the stem member such that the second opening abuts an interior bone tissue corresponding to a lateral cortex of the femur when the stem member extends into the femur.

6. The hip prosthesis of claim 1, further comprising a locking member configured to extend through the channel, wherein the locking member comprises a head portion that is matable with the first opening and a tip portion that protrudes outside of the second opening when the locking member is disposed in the channel, wherein the tip portion of the locking member is configured to penetrate the femur.

7. An assembly comprising:
a femoral component comprising a stem member configured to extend into a femur of a patient; and
a locking member configured to extend through the femoral component to affix the stem member to the femur.

8. The assembly of claim 7, wherein the stem member comprises a channel configured to receive the locking member, wherein the channel comprises a first opening and a second opening that is distal to the first opening, the channel has a longitudinal axis that intersects a longitudinal axis of the stem member.

9. The assembly of claim 8, wherein the femoral component further comprises a neck member extending from a proximal end portion of the stem member, and the first opening is located on the proximal end portion of the stem member and is spaced lateral relative to the neck member.

10. The assembly of claim 9, wherein the second opening is located on a lateral side of the stem member such that it abuts an interior bone tissue corresponding to a lateral cortex of the femur when the stem member extends into the femur.

11. The assembly of claim 8, wherein the locking member is longer than the channel such that, when the stem member extends into the femur, the locking member can be inserted into the channel through the first opening and a tip portion of the locking member can protrude outside of the second opening and penetrate the femur.

12. The assembly of claim 8, further comprising a guide member that is configured to matingly engage the channel, wherein the guide member comprises a passage that is longitudinally aligned with the channel when the guide member engages the channel such that a drill member can extend through the channel and the passage.

13. The assembly of claim 8, wherein the locking member comprises a head portion that is configured to threadably couple to the first opening of the channel.

14. The assembly of claim 8, wherein the locking member comprises a head portion and a threaded body portion extending from the head portion.

15. The assembly of claim 8, wherein the locking member comprises a head portion and a body portion extending from the head portion, wherein the body portion comprises a threaded portion and a non-threaded portion.

16. A method for implanting a hip prosthesis, the method comprising:
inserting a stem member of a femoral component into a femur of a patient; and
inserting a locking member through a channel of the stem member, the locking member being longer than the channel such that a tip portion of the locking member penetrates the femur.

17. The method of claim 16, wherein the channel comprises a first opening and a second opening that is distal to the first opening, the channel has a longitudinal axis that intersects a longitudinal axis of the stem member.

18. The method of claim 17, wherein the femoral component further comprises a neck member extending from a proximal end portion of the stem member, and the first opening is located on the proximal end portion of the stem member and is spaced lateral relative to the neck member.

19. The method of claim 17, wherein the second opening is located on a lateral side of the stem member such that it abuts an interior bone tissue corresponding to a lateral cortex of the femur when the stem member is inserted into the femur.

20. The method of claim 16, further comprising coupling a guide member to the channel, wherein the guide member comprises a passage that is longitudinally aligned with the channel when the guide member is coupled to the channel.
